(19) United States
(12) Patent Application Publication
(10) Pub. No.: US 2005/0106221 A1
(43) Pub. Date: May 19, 2005

(54) Kit for the treatment of a corn or a callus

(76) Inventor: Alan E. Nash, Canton, MA (US)

Correspondence Address: STEVEN N. FOX, ESQ., P.O. BOX 251, CANTON, MA, 02021, US

(21) Appl. No.: 10/713,824

(22) Filed: Nov. 14, 2003

(51) Int. Cl. A61K 9/70

(52) U.S. Cl. 424/443

(57) ABSTRACT

The present invention is a kit for use by a person to apply a daily treatment of medication and therapy to remove a corn or callus from the person's skin while providing maximum comfort to the person and minimal damage to healthy skin. In one embodiment, the kit comprises a box, an AM treatment system, and a PM treatment system for application at night. The AM treatment system comprises a plurality of AM foam bandages each having a medicated foam pad of hydrocolloid. The PM treatment system comprises a plurality of PM foam protective covers and a plurality of PM medicated non-woven patches impregnated with salicylic acid. The PM treatment system allows the person to remove a single PM medicated patch, trim it to fit the shape of the corn, and position it as desired over the corn. The person may then remove a single PM foam protective cover and apply it over the PM medicated patch, covering it completely, thereby limiting migration of the salicylic acid to healthy skin. Upon removal of the PM foam protective cover, the PM medicated patch along with a portion of the dead skin is removed neat and clean. The treatment cycle begins anew, wherein the AM foam bandage has the opportunity to heal any healthy skin that may have inadvertently come into contact with the salicylic acid of the PM treatment system as well as softening additional dead skin of the corn prior to the application of the next PM treatment system.

### FIELD OF THE INVENTION

The present invention relates to treatments for the removal of a corn or callus from the skin of a person.

### BACKGROUND OF THE INVENTION

A variety of non-prescription treatments have been developed to remove a corn or a callus from the skin of a person. Corns and calluses are dead, hardened skin. As such walking on one is the equivalent having a hard, thick coin underfoot, which would be very painful. Many conventional treatments have several drawbacks. By way of example only, one conventional treatment is sold under the brand name Dr. Scholl's® One Step Corn Removers. For example, such conventional treatments promote and require the user to wear said device continuously for forty-eight (48) hours wherein salicylic acid comes into contact with both the dead skin of the corn as well as the healthy skin of the user. The salicylic acid indiscriminately burns all the skin it comes into contact with. Obviously this is not desirable nor does it provide the consumer with an effective, gentle and safe product that can be easily stored and used as needed. The user is burning their skin while awake and active as well as when they are asleep and inactive. A further drawback is such conventional devices do not pre-treat or condition the corn or callus prior to application of the salicylic acid. A further drawback is that such devices are continuous in their application of acid and therefore cannot give the corn and or callus and more importantly the affected healthy skin around said area an opportunity to heal until the entire treatment has been administered, at which time the damage done to healthy skin may in fact be considerable.

### SUMMARY OF THE INVENTION

One object of the present invention is to provide maximum comfort to the user and, minimal damage to health skin, while effectively removing painful corns and calluses, utilizing a gentle 2-step process.

The present invention is a kit for use by a person to apply a daily treatment of medication and therapy to remove a corn or a callus from the person's skin while providing maximum comfort to the user and minimal damage to healthy skin. In one embodiment, the kit comprises a box having a decorative design thereon. The kit further comprises an AM treatment system and a PM treatment system removably stored within the box.

The AM treatment system comprises a plurality of AM foam bandages each having an upper surface and a lower surface, first and second end portions, and a median portion. In one embodiment, six (6) AM foam bandages are provided within the box. Each AM foam bandage comprises an adhesive coating disposed on the lower surface. Each AM foam bandage further comprises a medicated foam pad of hydrocolloid disposed on the adhesive coating at the median portion. The AM treatment system further comprises a release strip removably engaged along the adhesive surface on the lower surface of each AM foam bandage. The AM treatment system further comprises a plurality of AM packages adapted to individually seal the AM foam bandages for dispensing one at a time.

The PM treatment system comprises a plurality of PM foam protective covers and a plurality of PM medicated non-woven patches impregnated 40% by weight of salicylic acid. In one embodiment, six (6) PM foam protective covers and six (6) PM medicated patches are provided in the box. The PM medicated patch and the PM foam protective covers each comprise an upper and a lower surface. The PM medicated patches comprises a slightly tacky salicylic acid medicated coating layer. The PM medicated patches comprise a Mylar® backing strip engaged with one side of the slightly tacky salicylic acid medicated coating layer. In application, a plurality of PM medicated patches are disposed on a single Mylar® backing strip. This is carried in a single resealable package. Each of the PM foam protective covers comprise a foam film and an adhesive coating disposed on the lower surface of the foam film. Each of the PM foam protective covers comprise a Mylar® backing strip engaged with the adhesive coating to thereby allow the foam film to be removed from the backing strip one at a time. In application, a plurality of PM foam protective covers are disposed on a single Mylar backing strip. This is carried in a single reseal-able package.

The PM treatment system allows the person to remove a single PM medicated patch, trim it to fit the shape of the corn, and position it as desired over the corn. Then the person may remove a single PM foam protective cover and apply it over the PM medicated patch, covering it completely, which has the advantage of limiting migration of the salicylic acid. Furthermore when it's time to switch back to the AM treatment, upon removal of the PM foam protective cover, the PM medicated patch along with a portion of the dead skin is removed neat and clean. Then the cycle begins anew, wherein the AM foam bandage has the opportunity to start to heal any healthy skin that may have inadvertently come into contact with the salicylic acid of the PM treatment system as well as softening more of the dead skin of the corn prior to the application of the next PM treatment system. The AM packages and the first and second PM packages are different colors to facilitate use. The person may apply the AM treatment system to the corn or callus in the morning (or when active) and apply the PM treatment system at night (when ready to rest or inactive).

### BRIEF DESCRIPTION OF THE DRAWINGS

The following detailed description of the invention will be more fully understood with reference to the accompanying drawings in which:

FIG. 1 is a perspective view of the kit of the present invention showing the AM treatment system and the PM treatment system external of the box;

FIG. 2 is a perspective view of the AM foam bandage;

FIG. 3 is a perspective view of the PM foam protective cover;

FIG. 4 is a perspective view of the PM medicated patch;

FIG. 5 is a cross-section view of the AM foam bandage having the hydrocolloid taken along line 5-5 of FIG. 2;

FIG. 6 is a cross-section view of the PM medicated patch having the salicylic acid taken along line 6-6 of FIG. 4; and

FIG. 7 is a cross-section view of the PM foam protective cover taken along line 7-7 of FIG. 3.

### DETAILED DESCRIPTION OF THE PREFERRED EMBODIMENTS

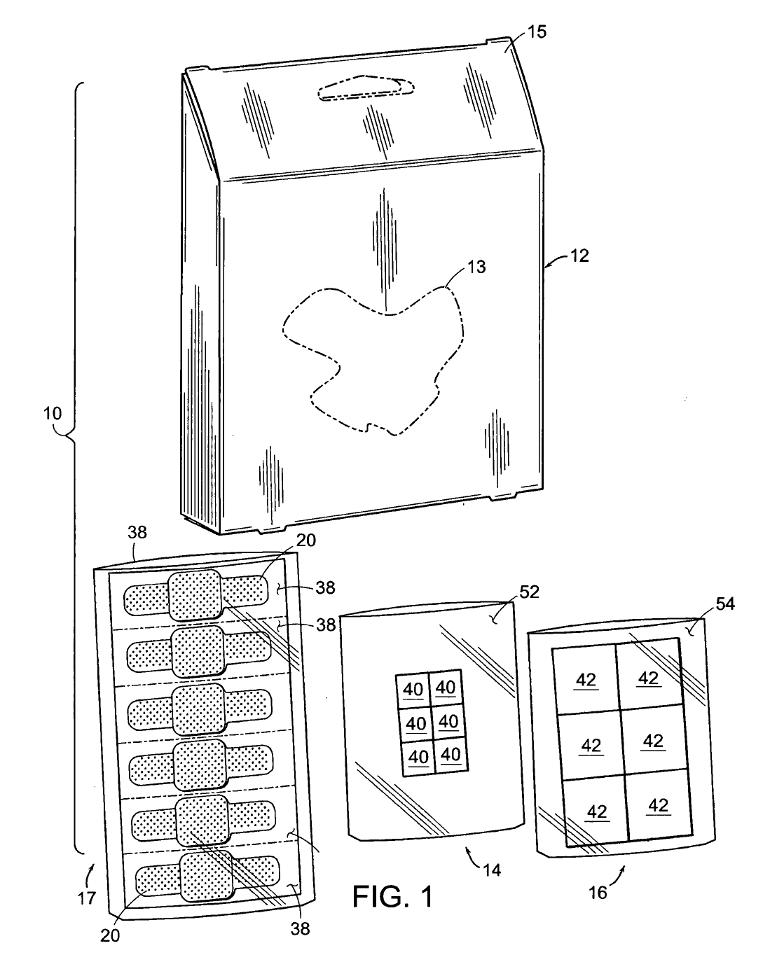

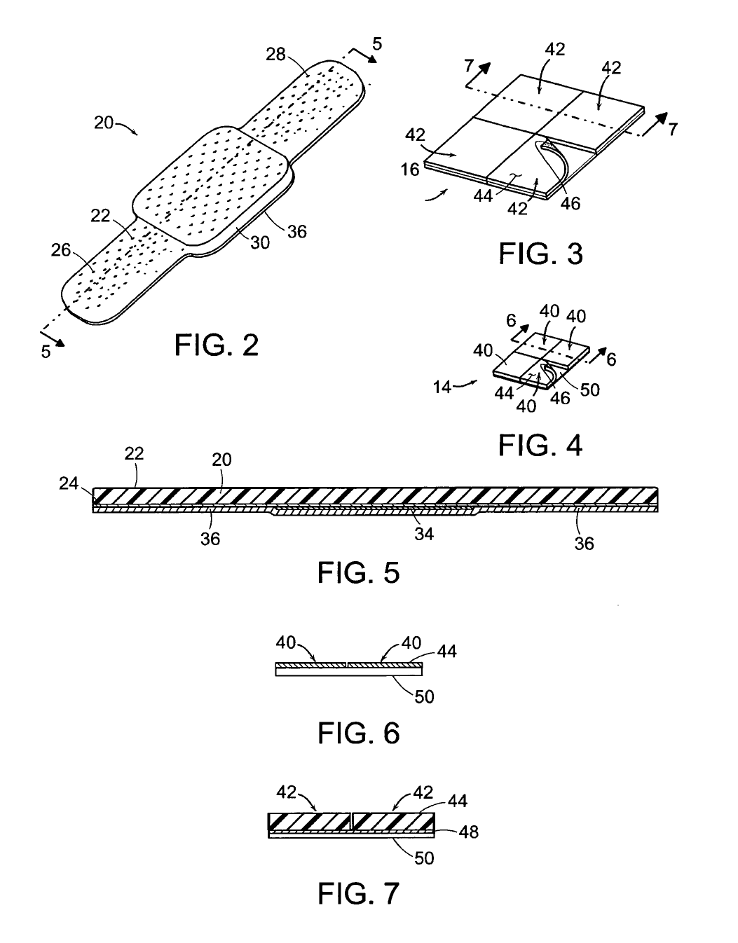

Referring to FIGS. 1-7, the present invention is a kit 10 for use by a person (not shown) to apply a daily treatment of medication and therapy to remove a corn or a callus (not shown) from the person's skin. In one embodiment, the kit 10 generally comprises a box 12 and an AM treatment system 17 and a PM treatment system 14 and 16 removably stored within the box 12.

The box 12 comprises a decorative design 13 and a cover 15 to open and close the box 12. The box may be made from a variety of conventional materials such as recycled paper. The box 12 may be made by a variety of conventional processes such as die cutting, film laminating, conventional printing.

The AM treatment system 17 comprises a plurality of AM foam bandages 20 each having an upper surface 22 and a lower surface 24, first and second end portions 26 and 28, and a median portion 30. In one embodiment, six (6) AM foam bandages 20 are provided within the box 12. Each AM foam bandage 20 comprises an adhesive coating 32 disposed on the lower surface 24. Each AM foam bandage 20 further comprises a medicated foam pad 34 of hydrocolloid disposed on the adhesive coating 32 at the median portion 30. The AM treatment system 17 further comprises a release strip 36 removably engaged along the adhesive surface 32 on the lower surface 24 of each AM foam bandage 20. The AM treatment system 17 further comprises a plurality of AM packages 38 adapted to individually seal the AM foam bandages 20 for dispensing one at a time. The AM foam bandages 20 are made from a roll of foam and carboxyl methyl cellulose. The AM foam bandages 20 can be die cut from the roll in various shapes and sizes and sealed with the AM package 38 by conventional bandage packaging processes and multiple cut-away strips.

The PM treatment system 14 and 16 comprises a plurality of PM foam protective covers 42 and a plurality of PM medicated non-woven patches 40 impregnated 40% by weight of salicylic acid. In one embodiment, six (6) PM foam protective covers 42 and six (6) PM medicated patches 40 are provided in the box 12. The PM medicated patches 40 and PM foam protective covers 42 each comprise an upper surface 44 and a lower surface 46. The PM medicated patches 40 comprise a slightly tacky salicylic acid medicated coating layer. The PM medicated patches 40 comprise a Mylar® backing strip 50 engaged with one side 46 of the slightly tacky salicylic acid medicated coating layer. In application, a plurality of PM medicated patches 40 are disposed on a single Mylar® backing strip 50. This is carried in a single resealable package 52. Each of the PM foam protective covers 42 comprise a foam film and an adhesive coating 48 disposed on the lower surface 46 of the foam film. The PM foam protective covers 42 comprise a Mylar® backing strip 50 engaged with the adhesive coating 48 to thereby allow the foam film to be removed from the backing strip 50 one at a time. In application, a plurality of PM foam protective covers 42 are disposed on a single Mylar® backing strip 50. This is carried in a single reseal-able package 54. The AM packages 38 and the first and second PM packages 52 and 54 are preferably made of different colors to facilitate use and recognition by the person. By way of example only, the AM packages 38 may be white and the first and second PM packages 52 and 54 may be brown and green, respectively.

The PM treatment system 14 and 16 allow the person to remove a single PM medicated patch 40, trim it to fit the shape of the corn, and position it as desired over the corn. Then the person may remove a single PM foam protective cover 42 and apply it over the PM medicated patch 40, covering it completely, which has the advantage of limiting migration of the salicylic acid. Furthermore when it is time to switch back to the AM treatment, upon removal of the PM foam protective cover 42, the PM medicated patch 40 along with a portion of the dead skin is removed neat and clean. Then the cycle begins anew, wherein the AM foam bandage 20 has the opportunity to start to heal any healthy skin that may have inadvertently come into contact with the salicylic acid of the PM treatment system 14 and 17 as well as softening more of the dead skin of the corn prior to the application of the next PM treatment system 14 and 16. The person may apply the AM treatment system 17 to the corn or callus in the morning (or when otherwise active) and apply the PM treatment system 14 and 16t at night (when ready to rest or otherwise inactive).

The PM medicated patches 40 are made from foam. The PM medicated patches 40 can be die cut from the roll in various shapes and sizes and sealed with the first PM package 52 by conventional bandage packaging processes. The PM foam protective covers 42 are likewise made from foam. The PM foam protective covers 42 can be die cut from the roll in various shapes and sizes and sealed with the first PM package 54 by conventional bandage packaging processes.

The foregoing description is intended primarily for purposes of illustration. This invention may be embodied in other forms or carried out in other ways without departing from the spirit or scope of the invention. Modifications and variations still falling within the spirit or the scope of the invention will be readily apparent to those of skill in the art.

1. A kit for use by a person to apply a daily treatment of medication and therapy to remove a corn or callus from the person's skin, the kit comprising:
(a) a box having a decorative design thereon;
(b) an AM treatment system removably disposed within said box; said AM treatment system comprises a plurality of AM foam bandages each having a medicated pad of hydrocolloid; said AM treatment system further comprises a release strip removably engaged with each of said AM foam bandages;
(c) a PM treatment system removably disposed within said box; said PM treatment system comprises a plurality of PM foam protective covers and a plurality of PM medicated patches having salicylic acid; said PM treatment system further comprises first and second backing strips engaged with said PM foam protective covers and said PM medicated patches, respectively; and
whereby the person may apply the AM treatment system to the corn or callus when active such as in the morning and apply the PM treatment system when inactive such as at night.

2. The kit of claim 1, wherein said M treatment system comprises an AM package adapted to individually seal said AM foam bandages.

3. The kit of claim 2, wherein said PM treatment system comprises first and second PM packages to seal said PM medicated patches and said PM foam protective covers, respectively.

4. The kit of claim 4, wherein said PM medicated patches are 40% by weight of salicylic acid.

5. The kit of claim 4, wherein said AM package and said first and second PM packages are different colors.

6. The kit of claim 5, wherein each of said AM and first and second PM packages are made from paper.

7. The kit of claim 6, wherein said AM foam bandage is made from foam.

8. The kit of claim 7, wherein said PM foam protective cover is made from foam.
